(19) United States
(12) Patent Application Publication
(10) Pub. No.: US 2005/0197585 A1
(43) Pub. Date: Sep. 8, 2005

(54) Vascular blood pressure monitoring system with transdermal catheter and telemetry capability

(75) Inventors: Brian P. Brockway, Shoreview, MN (US); Perry A. Mills, Arden Hills, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Transoma Medical, Inc., St. Paul, MN (US)

(21) Appl. No.: 11/073,413

(22) Filed: Mar. 4, 2005

Related U.S. Application Data
(60) Provisional application No. 60/550,764, filed on Mar. 6, 2004.

(51) Int. Cl. A61B 5/02; A61B 5/00

(52) U.S. Cl. 600/486; 600/485; 600/561

(57) ABSTRACT

Systems and methods for monitoring intravascular pressure of a patient. The system includes a pressure sensor and catheter assembly having a pressure sensor portion and a transdermal catheter portion. The transdermal catheter portion has a fluid filled lumen and a distal barrier to maintain patency. A transmitter unit may be connected to the pressure sensor portion via a lead. The transmitter unit transmits pressure data to a remote receiver unit that may communicate (directly or indirectly) with a cable management device, a data acquisition device, a monitoring instrument, a computer, a modem, a telecommunication line, a network, etc. In addition, the system may include means to correct for pressure variations due to any difference in elevation between the sensor assembly and the heart.

### FIELD OF THE INVENTION

The present invention generally relates to devices, systems and methods for monitoring intravascular pressure.

### BACKGROUND OF THE INVENTION

For various therapeutic and/or diagnostic reasons, some patients, particularly those in the intensive care and coronary care units (ICU and CCU) of a hospital, require monitoring of certain blood parameters such as blood chemistry and blood pressure. To accommodate this need, an arterial or venous line may be inserted into the patient's vascular system to permit blood draws and blood pressure monitoring. For example, a line may be inserted into the patient's radial artery near the wrist.

The pressure measured using the line is transmitted via a cable to a computer system which stores and displays systolic, diastolic and waveform pressure data. Depending on the location of the computer, the cable may be draped across the patient or otherwise extend from the patients arm to a bedside location.

To assure the accuracy of pressure measurements, and to assure the ability to draw blood, the line must remain patent. To maintain patency, the line may be connected via tubing to a pressurized bag for continuous infuision of heparinized saline or the like. Depending on the location of the pressurized bag, the tubing may be draped across the patient or otherwise extend from the patients arm to a bedside location. If the patient is allergic to heparin, continuous infusion is undesirable, so the line must be manually flushed on a periodic basis.

Despite such efforts to maintain patency, it is not uncommon for the line to become obstructed, and for pressure measurements to be off as much as 30 mmHg. As such, pressure measurements are periodically taken using a pressure cuff to confirm the accuracy of pressure measurements taken using the line.

The computer cable and the tubing often interfere with efforts to move or reposition the patient. Even if tubing is not used (i.e., a manual flush is used), the need to manually flush the line is additionally burdensome, and the computer cable still interferes with efforts to move the patient and to allow the patient to walk in the hallways during the recovery process. Furthermore, the need to periodically take pressure measurements using a pressure cuff represents yet another burden. Thus, there is a need for a pressure monitoring system that eliminates the burdens outlined above.

### SUMMARY OF THE INVENTION

To address this need, various embodiments are described herein which provide a blood pressure monitoring system that utilizes a sensor and catheter assembly having a fluid-filled transdermal catheter to maintain patency, and a transmitter unit for wireless connection to a computer system via a remote receiver unit. Because the fluid-filled transdermal catheter maintains patency over long periods of time, the need for connection to a bag of heparinized saline is eliminated, and the need to confirm accuracy using a pressure cuff is eliminated. In addition, because the transmitter unit provides for wireless communication, the need for connection via a computer cable is eliminated.

Thus, the various embodiments described herein provide a system for monitoring intravascular blood pressure that is easier to use because it eliminates tubes and cables, and is more reliable because it maintains patency over long periods of time. Further, it allows the patient to ambulate much more easily and conveniently. Alternative embodiments provide for direct connection of the pressure sensor to a monitoring instrument or the like, thus eliminating the need for telemetry.

The sensor and catheter assembly may be releasably connected to the transmitter unit via an electric lead, or may be combined into a single unit. To be cost effective while minimizing the risk of infection, the sensor and catheter assembly may be disposable, and the transmitter unit may be reusable. The sensor and catheter assembly may be taped or otherwise connected to the patient by conventional means, and the transmitter unit may include a strap, clip or the like for connection to the patient's body (e.g., arm, chest, etc.) or clothing. The transmitter unit transmits pressure data to a remote receiver unit that may communicate (directly or indirectly) with a cable management device, a data acquisition device, a monitoring instrument device, a computer, a modem, a telecommunication line or system, a network, etc. In addition, the system may include means to correct for pressure variations due to any difference in elevation between the sensor and catheter assembly and the heart.

### BRIEF DESCRIPTION OF THE DRAWINGS

FIG. 1 is a schematic plan view of a system for monitoring intravascular pressure of a patient;

FIG. 2 is a schematic block diagram of a sensor and catheter assembly and a transmitter unit for use in the system illustrated in FIG. 1;

FIGS. 2A-2C are lateral cross-sectional views of the transdermal catheter taken along line 2-2 in FIG. 2;

FIG. 2D is a longitudinal cross-sectional view of the transdermal catheter in FIG. 2;

FIG. 3 is a schematic block diagram of a receiver unit connected to a cable management and data acquisition device for use in the system illustrated in FIG. 1; and

FIGS. 4-7 illustrate various alternative systems, components and methods to detect and/or correct for pressure variations due to any difference in elevation between the heart and the sensor and catheter assembly.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention.

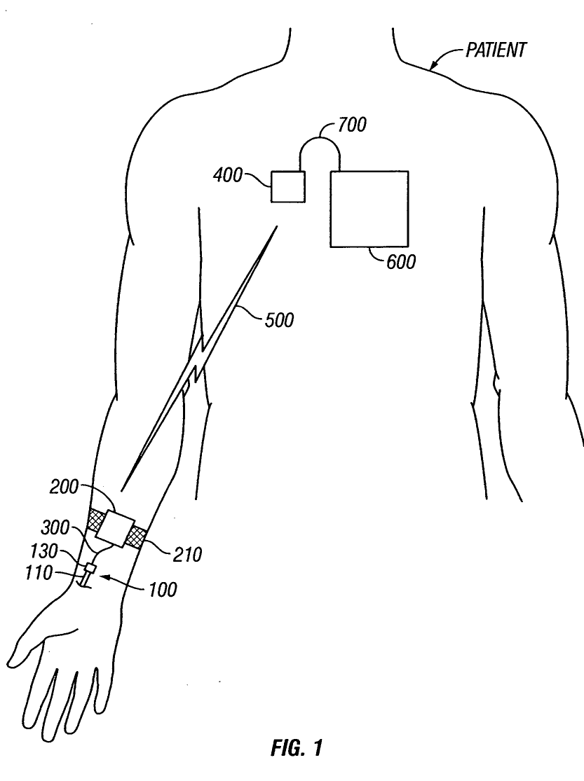

With reference to FIG. 1, a system for monitoring intravascular pressure of a patient is schematically shown. In this illustrative embodiment, the system includes a pressure sensor and catheter assembly 100 connected to a transmitter unit 200 by lead 300. The transmitter unit 200 receives blood pressure signals from the sensor and catheter assembly 100 via lead 300. To be cost effective while minimizing the risk of infection, the sensor and catheter assembly 100 may be disposable, and the transmitter unit 200 may be reusable. Thus, it may be desirable to incorporate the majority of components in the reusable transmitter unit 200, leaving a minority of components in the disposable sensor and catheter assembly 100. However, those skilled in the art will recognize that the arrangement and allocation of components between the transmitter unit 200 and the sensor and catheter assembly 100 are described herein by way of example, not limitation, and that changes may be made accordingly. For example, the pressure sensor and catheter assembly 100 may be combined with the transmitter unit 200, thus eliminating lead 300.

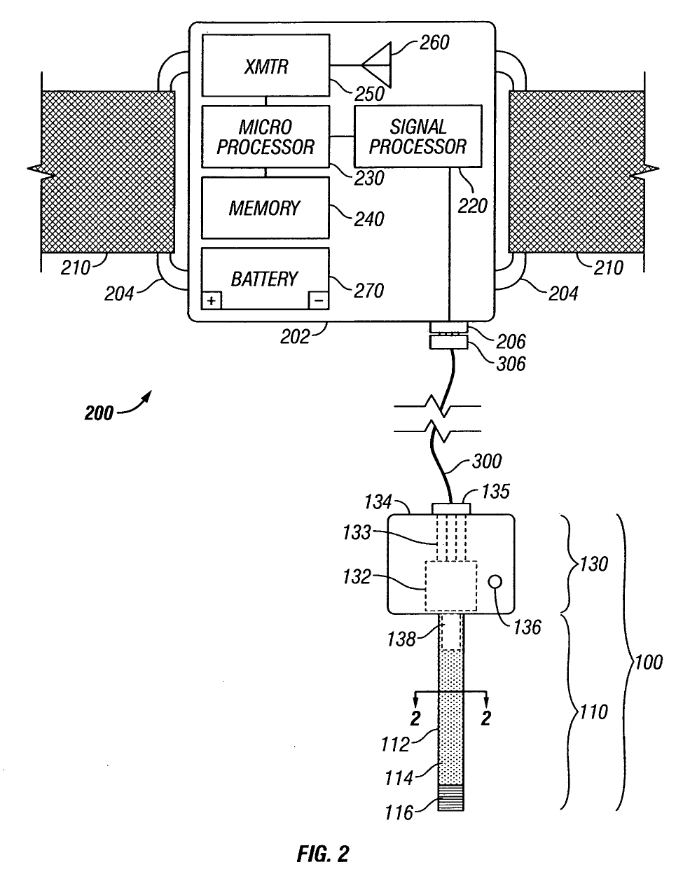

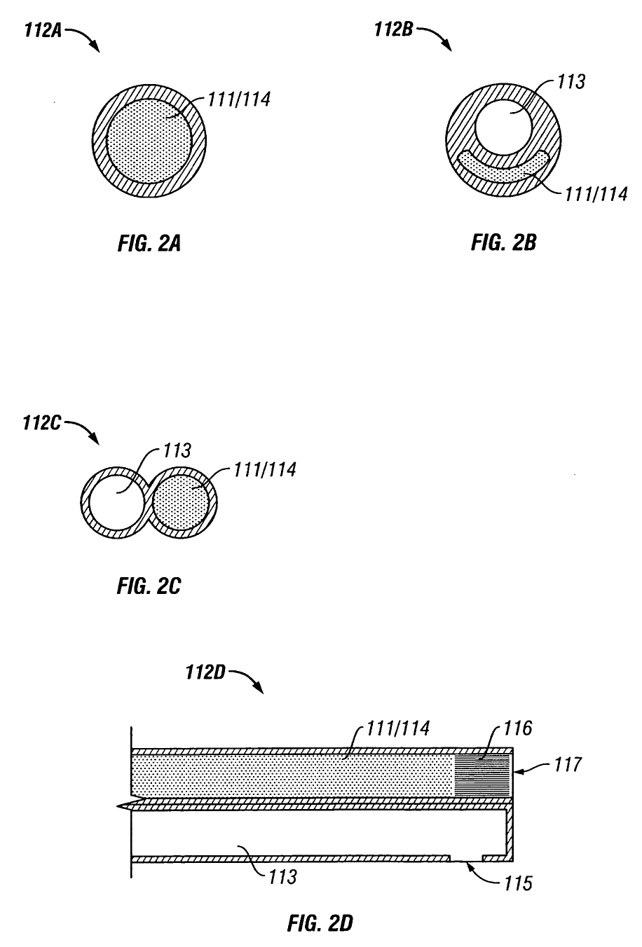

The pressure sensor and catheter assembly 100 includes a transdermal catheter portion 110 connected to a pressure sensor portion 130. The transdermal catheter portion 110 is filled with a pressure transmitting fluid and includes a distal barrier such as a viscous plug or compliant membrane, which are described in more detail with reference to FIG. 2. The fluid-filled transdermal catheter portion 110 remains patent over long periods of time when inserted into a vascular lumen, thereby eliminating the need for flushing and/or the need for connection via tubing to a bag of heparinized saline. This reduces the burden on nursing staff and makes it easier to move the patient or otherwise change the patient's position. The long-term patency of the fluid-filled transdermal catheter portion 110 also eliminates the need to confirm accuracy using a pressure cuff.

The system also includes a receiver unit 400 which communicates with the transmitter unit 200 by a wireless link 500. The wireless link may comprise radio telemetry, infrared telemetry, inductive coupling, passive transponders, or using the body as a conductor (referred to as “intracorporeal conductive communication” or a “personal area network”), for example. For purposes of illustration, not limitation, the following description is given with reference to radio telemetry as the mode of wireless communication. The wireless link 500 eliminates the need for connection via a cable, thereby making it easier to move the patient or otherwise change the patient's position.

Alternatively, the transmitter unit 200 and the receiver unit 400 may be eliminated in favor of a direct hardwire connection between a monitoring instrument and the sensor and catheter assembly 100. In this alternative embodiment, the pressure sensor portion 130 may be configured for analog interface with a conventional pressure monitoring instrument according to AAMI (Association for the Advancement of Medical Instrumentation) standard BP22.

The receiver unit 400 may be connected (directly or indirectly) to a cable management device, a data acquisition device, a monitoring instrument, a computer, a modem, a network, a telecommunication line, or the like, for example. In the illustrated embodiment, the receiver unit 400 is connected to a combined cable management system and data acquisition device 600, an example of which is described in U.S. Pat. No. 6,533,723 to Lockery et al., the entire disclosure of which is incorporated herein by reference. The combined cable management system and data acquisition device 600 may be connected, in turn, to a patient monitoring device, a computer, a modem, a network, or the like, to monitor, display, analyze and/or communicate pressure data obtained from the sensor and catheter assembly 100 via transmitter unit 200 and receiver unit 400.

The transmitter unit 200, the receiver unit 400 and/or device 600 may alternately incorporate transceivers. For example, device 600 may retransmit the signal from receiver unit 400 to a monitoring station where the blood pressure signal is observed and recorded. Additionally or alternatively, link 500 may be bi-directional such that unit 400 or device 600 communicates with unit 200, enabling unit 400 or device 600 to signal unit 200 to initiate data transmission. In this embodiment, unit 200 may save blood pressure data for some period of time (e.g. 15 seconds) and then transmit the data in a burst to unit 400. This approach conserves power when using a higher frequency telemetry link that can transmit data at a higher rate, but may be limited to a low duty cycle because of the power consumption required for a higher frequency link.

The transdermal catheter portion 110 extends through the patient's skin and into a vascular lumen, such as into an artery or vein. The transdermal catheter 110 may be introduced into the vascular lumen using conventional techniques such as a splittable butterfly style needle. With this technique, the needle is inserted into the vessel, the transdermal catheter 110 is inserted into the lumen of the needle, the needle is retracted and then split apart to remove it from the transdermal catheter 110. Alternatively, the transdermal catheter 110 may comprise a rigid polymeric or metallic (e.g., stainless steel) construction to enable the transdermal catheter 110 to be directly inserted into the vascular lumen. In the illustrated embodiment, the transdermal catheter portion 110 is shown accessing the radial artery near the wrist. However, it will be appreciated that the transdermal catheter portion 110 may access a wide variety of arteries and veins, preferably near the dermal surface, such as the brachial artery, the brachial vein, the subclavian artery, the subclavian vein, the femoral artery, the femoral vein, etc., to measure intravascular pressure.

The sensor and catheter assembly 100 may be connected to the patient's body adjacent the access site by a disposable connector such as adhesive tape or the like. The transmitter unit 200 may be connected to the patient's body or clothing proximate the access site with a reusable connector 210. The receiver unit 400 may be located proximate the combined cable management system and data acquisition device 600, which is typically mounted near the patient's chest. The receiver unit 400 may be connected to the patient's body or clothing in a manner similar to transmitter unit 200, or may be secured to the cable management system and data acquisition device 600. In some embodiments, the receiver unit 400 may be positioned near the heart for correction of differences in elevation between the heart and the sensor and catheter assembly 100.

With reference to FIG. 2, details of the sensor and catheter assembly 100 and the transmitter unit 200 are schematically illustrated. The sensor and catheter assembly 100 includes the transdermal catheter portion 110 connected to the pressure sensor portion 130. The pressure sensor portion 130 includes a pressure transducer 132 contained in housing 134. A passage 136 such as a hole (shown) or flexible diaphragm may be used to provide pressure communication through the housing 134 such that the pressure transducer 132 may make intravascular pressure measurements relative to barometric pressure. Alternatively, intravascular pressure measurements may be made relative to vacuum, in which case a barometric pressure correction scheme may be utilized. A nipple tube 138 extends from the housing 134 facilitates connection of the transdermal catheter portion 110 and is in fluid communication with the pressure transducer 132.

The pressure transducer 132 may be of the piezoresistive, resonant structure, or capacitive type, for example. For example, pressure transducer may comprise a piezoresistive Wheatstone bridge type silicon strain gauge available from Sensonor of Horton, Norway. The pressure transducer 132 is connected to lead 300 by wires 133 and connector block 135. For further information and other aspects pertinent to the pressure sensor portion 200, reference may be made to U.S. patent application Ser. No. 10/717,179, filed Nov. 17, 2003, entitled Implantable Pressure Sensors, the entire disclosure of which is incorporated herein by reference.

An electronics module (not shown) may be disposed in the housing 134 of the pressure sensor portion 130 to provide excitation to the pressure transducer 132, amplify the pressure signal, and/or digitally code the pressure information for communication to the transmitter unit 200 via the flexible lead 300. The electronics module may also provide for temperature compensation of the pressure transducer 132 and provide a calibrated pressure signal. A temperature measurement device (not shown) may be included within the electronics module to compensate the pressure signal from temperature variations. The electronics module and the temperature measurement device may alternatively be incorporated into the transmitter unit 200 to reduce the cost of the sensor and catheter assembly 100 such that the sensor and catheter assembly 100 may be disposable with less wasted expense.

As described previously, the transmitter unit 200 and the receiver unit 400 may be eliminated in favor of a direct hardwire connection between a monitoring instrument and the sensor and catheter assembly 100. In this alternative embodiment, the pressure sensor portion 130 may be configured for analog interface with a conventional pressure monitoring instrument according to AAMI (Association for the Advancement of Medical Instrumentation) standard BP22. For this application, the pressure transducer 132 preferably has a minimum sensitivity of 5 uV/V/mmHg, a pressure range of −30 to 300 mmHg, an excitation impedance greater than 200 Ohms, an unbalance of less than ±75 mmHg (to allow the unbalance to be compensated for by the monitoring instrument), and an accuracy of 1 mmHg+1% of reading for −20 to 50 mmHg and ±3% of reading for 50 to 300 mmHg.

The transdermal catheter portion 110 refers pressure from the intravascular pressure measurement site to the pressure transducer 132 located inside the housing 134. The transdermal catheter portion 110 may comprise a tubular shaft 112 filled with a pressure transmitting fluid 114 and including a distal barrier 116 such as a viscous plug (shown) or flexible membrane. The size (length and diameter) of the transdermal catheter portion 110 will vary depending on the blood vessel being accessed and the thickness of the dermal layer at the access site (i.e., the depth of the blood vessel from the dermal surface). For example, the transdermal catheter portion 110 may have a diameter of approximately 0.5 to 1 mm and a length of approximately 2 cm -3 cm to access the radial artery near the wrist. The size of the catheter portion 110 may be minimized to reduce the possibility of morbidity and to reduce patient discomfort during insertion.

The transdermal catheter portion 110 may comprise a wide variety of materials, constructions and dimensions. For example, to prevent inadvertent kinking and/or collapse, the tubular shaft 112 may comprise a coil or braid embedded in a polymeric tube. Alternatively, the tubular shaft 112 may comprise a metallic hypotube to facilitate insertion without an introducer. Various tapers, flares, wall thicknesses, etc. may also be incorporated into the transdermal catheter portion 110. Various materials and construction alternatives for the transdermal catheter portion 110 are described in U.S. patent application Ser. No. 10/077,566, filed Feb. 15, 2002, entitled Devices, Systems and Methods for Endocardial Pressure Measurement, and U.S. patent application Ser. No. ______, filed ______, entitled Pressure Transmission Catheter for Implantable Pressure Sensors, which claims the benefit of U.S. Provisional Patent Application No. 60/454823, filed Mar. 12, 2003, the entire disclosures of which are incorporated herein by reference. The transdermal catheter portion 110 may optionally include one or more EGM electrodes or other physiological sensors as described in U.S. Pat. No. 6,296,615 to Brockway et al., the entire disclosure of which is incorporated herein by reference.

The transdermal catheter portion 110 refers pressure from the intravascular lumen to the pressure transducer 132 via a pressure transmitting fluid 114 and a pressure responsive barrier 116, both contained in the lumen of the tubular shaft 112. The barrier 116 may comprise a gel plug and/or flexible membrane disposed in or over the distal opening of the tubular shaft 112 to isolate the liquid-filled lumen from bodily fluids and to retain the fluid 114 in the lumen without impeding pressure transmission therethrough. In one embodiment, the fluid 114 is chosen to be a fluorinated silicone oil and the barrier 116 is chosen to be dimethyl silicone gel. Further aspects of suitable fluids and gels are described in U.S. Pat. No. 4,846,191 to Brockway et al., and U.S. patent application Ser. No. 10/272,489, filed Oct. 15, 2002, entitled Improved Barriers and Methods for Pressure Measurement Catheters, the entire disclosures of which are incorporated herein by reference.

With reference to FIGS. 2A-2D, transverse cross-sectional views of the tubular shaft 112 are shown in FIGS. 2A-2C, and a longitudinal cross-sectional view is shown in FIG. 2D. In a first embodiment shown in FIG. 2A, the catheter shaft 112A comprises a circular cross-section with a circular lumen 111 containing the fill-fluid 114 as described above. In a second embodiment shown in FIG. 2B, the catheter shaft 112B comprises a circular outer cross-section with a first (e.g., annular) lumen 111 containing the fill-fluid 114 for pressure measurement and a second (e.g., circular) lumen 113 for drawing blood or injecting fluids into the vascular lumen of the patient. The tubular shaft 112B may comprise a polymeric material capable of being pierced by a hypodermic needle for the injection of fluids or withdrawal of blood therethrough. In a third embodiment shown in FIG. 2C, the catheter shaft 112C comprises two side-by-side lumens, with a first (e.g., circular) lumen 111 containing the fill-fluid 114 for pressure measurement and a second (e.g., circular) lumen 113 for drawing blood or injecting fluids into the vascular lumen of the patient. The portion of the tubular shaft 112C defining lumen 113 may comprise a polymeric material capable of being pierced by a hypodermic needle as described above. For all embodiments described herein, the distal openings of the lumens 111/113 may face distally or laterally. For example, as shown FIG. 2D, the first (pressure measurement) lumen 111 may have a distal-facing opening 117, and the second (blood draw) lumen 113 may have a lateral-facing opening 115. The combination of a distal-facing opening 117 of the first lumen 111 and a lateral-facing opening 115 of the second lumen 113 reduces the likelihood that blood clots, thrombus or the like associated with the blood draw lumen 113 will interfere with the pressure measurement lumen 111. These embodiments of the lumen structure of the catheter shaft 112 are given by way of example, not necessarily limitation.

The transmitter unit 200 includes a housing 202, which may contain the shown internal components, in addition to other components not shown such as the electronics module and temperature sensor described previously. An electrical plug receptacle 206 may be externally connected to the housing 202 to receive a mating electrical plug 306 of the flexible lead 300. The plug 306 and plug receptacle 206 may comprise a wide variety of releasable electrical connectors known in the art. The releasable electrical connection 206/306 allows the transmitter unit 200 to be readily connected and disconnected from the sensor and catheter assembly 100, thereby permitting reuse of the transmitter unit 200 after disposal of the sensor and catheter assembly 100.

The sensor portion 130 is electrically coupled to a signal processor 220 via lead 300 and associated electrical connections. The signal processor 220 may perform some or all of the same functions as the electronics module described previously, in addition to other signal processing functions such as filtering, A/D conversion, amplification, etc. The signal processor 220 may be connected to a microprocessor 230 and associated memory device 240. The memory device 240 may store pressure data, threshold data, software for execution by the microprocessor 230, etc. to enable a wide variety of functions. Microprocessor 230 may perform signal analysis, signal encoding, etc., and a transmitter (or transceiver as described previously) 250 with associated antenna 260 may be connected to the microprocessor 230 for transmission of pressure data to receiver unit 400. The blood pressure data signal may either be transmitted in real time from the transmitter unit 200 to the receiver unit 400, or it may be stored for a period of time in memory 240 and transmitted as a burst to receiver unit 400 as described previously. When stored in memory 240, various compression techniques may be applied to the blood pressure data signal to reduce the amount of data to be telemetered to receiver unit 400. In addition, it may be advantageous to incorporate error detection and correction capability in the receiver unit 400, such that transmitted data may be analyzed by the receiver unit 400 to improve the reliability of the connection. In this embodiment, the receiver unit 400 may incorporate a transceiver as described previously, and if an error is detected, unit 400 can signal to unit 200 that the data packet was not received correctly and request retransmission. This approach may have advantages in reduction of power and improvement of reliability in the transmission link 500. A rechargeable and/or replaceable battery 270 provides electrical power to the internal components, and may provide electrical power to components contained in the pressure sensor portion 130 of the sensor and catheter assembly 100.

To facilitate connection of the transmitter unit 200 to the patient's body or clothing proximate the access site, a connector such as a strap 210 may be wrapped around a portion of the patient's body and secured to the housing 202 by strap bars 204. For example, if the access site is the radial artery near the wrist, the transmitter unit 200 may be connected to the patient's wrist by an elastic strap 210 wrapped around the wrist. The means for connection to the patient's body or clothing may vary depending on the chosen access site. For example, if the brachial artery or vein is selected as the access site, an elastic strap may be used to wrap around the patient's upper or lower arm near the elbow. Alternatively, if the femoral artery or vein is selected as the access site, an elastic strap may be used to wrap around the patient's thigh near the groin. As a further alternative, if the subclavian artery or vein is selected as the access site, an elastic strap may be used to wrap around the patient's shoulder, or a clip may be used to secure the transmitter unit to the patient's shirt. Those skilled in the art will appreciate that the means for connection to the patient's body or clothing will vary depending on the access site and may include, without limitation, a band, a strap, a belt, a clip, a necklace, a two piece connector such as matching Velcro surfaces, snaps, etc.

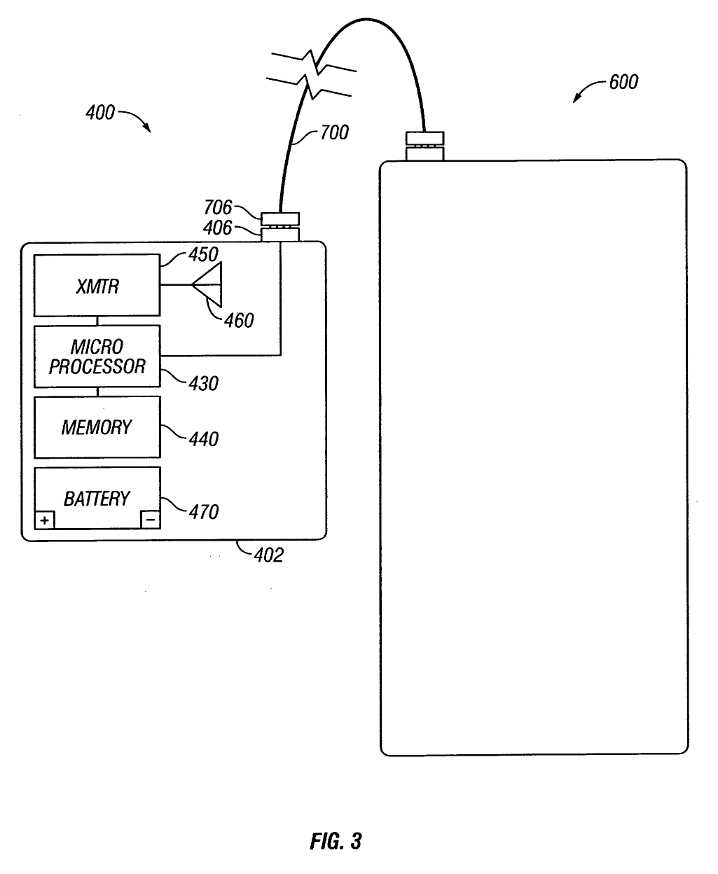

With reference to FIG. 3, details of the receiver unit 400 are schematically illustrated. The receiver unit 400 includes a housing 402, which may contain the shown internal components, in addition to other components as desired but not shown. An electrical plug receptacle 406 may be externally connected to the housing 402 to receive a mating electrical plug 706 of the flexible lead 700. The plug 706 and plug receptacle 406 may comprise a wide variety of releasable electrical connectors known in the art. The releasable electrical connection 406/706 allows the receiver unit 400 to be readily connected and disconnected from the combined cable management and data acquisition device 600 or other device such as a patient monitoring device, a computer, a network, etc.

A receiver (or transceiver as described previously) 450 with associated antenna 460 receives pressure data from transmitter unit 200. The receiver may be connected to a microprocessor 430 and associated memory device 440. The memory device 440 may store pressure data, threshold data, software for execution by the microprocessor 430, etc. to enable a wide variety of functions. Microprocessor 430 may perform signal analysis, signal decoding, error detection, error correction, etc., and transmits pressure data to device 600 via cable 700. A rechargeable and/or replaceable battery 470 provides electrical power to the internal components. It may likewise be advantageous to employ a closed telemetry unit where the device is disposed when the battery runs down.

Reference may be made to the following patents and patent applications for further information and other aspects pertinent to the pressure sensor and catheter assembly 100, the transmitter unit 200 and the receiver unit 400: U.S. patent application Ser. No. 10/077,566, filed Feb. 15, 2002, entitled Devices, Systems and Methods for Endocardial Pressure Measurement; U.S. Pat. No. 4,846,191 to Brockway et al.; U.S. Pat. No. 6,033,366 to Brockway et al.; U.S. Pat. No. 6,296,615 to Brockway et al.; and PCT Publication WO 00/16686 to Brockway et al., the entire disclosures of which are incorporated herein by reference. Reference may be made to the following patents and patent applications for further information and other aspects pertinent to the combiner cable management system and data acquisition device 600: U.S. Pat. No. 6,533,723 to Lockery et al. and U.S. patent application Publication No. 2003/0009106 to Sitzman et al., the entire disclosures of which are incorporated herein by reference

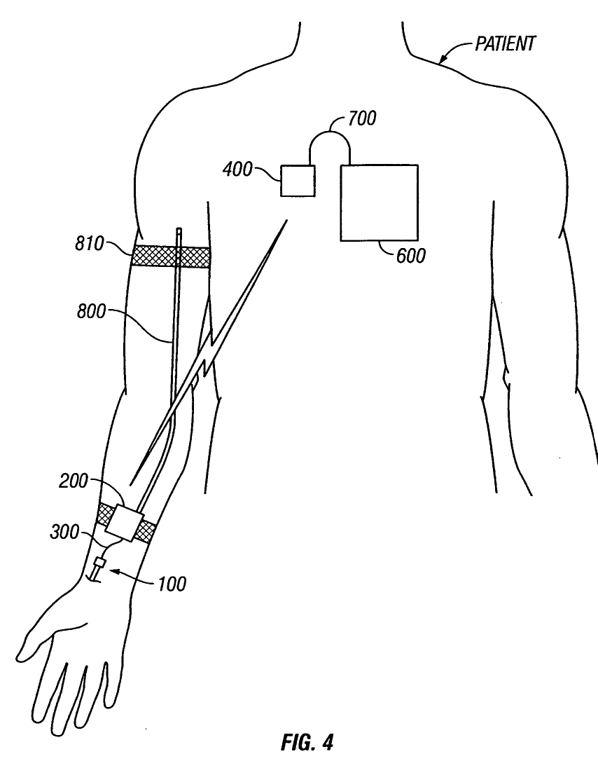

With general reference to FIGS. 4-7, various systems, components and methods are schematically illustrated which detect and/or correct for pressure variations due to any difference in elevation between the sensor and catheter assembly 100 and the heart of the patient. These embodiments address the difference in pressure that can occur when the elevation of the sensor and catheter assembly 100 is different than the elevation of the heart. For example, if the patient is standing with his/her arms at the side, and the sensor and catheter assembly 100 is positioned near the wrist accessing the radial artery, the sensor portion 200 will measure a pressure that is higher than blood pressure at the heart. Alternatively, if the patient is standing with his/her arms raised above the head, the sensor portion 200 will measure a pressure that is lower than blood pressure at the heart. Even if the patient is in the supine position (e.g., laying down flat on a bed), the sensor portion 200 will read a pressure that is the same as blood pressure at the heart only if the location of the arm where pressure is being measured is not raised or lowered with respect to the level of the heart. Thus, the following embodiments present schemes to address differences in pressure resulting from differences in elevation between the patient's heart and the catheter and sensor assembly 100.

With specific reference to FIG. 4, an alternative system for monitoring intravascular pressure of a patient is schematically shown. In this alternative system, most aspects are the same as or similar to the system illustrated in FIG. 1, except for the provision of a second fluid-filled catheter 800 and its associated components. The second catheter 800 refers pressure from a point in close proximity to the elevation of the secured to the patient or the patient's clothing using one or more reusable connectors 810, which may be similar to connector 210 described previously. With this arrangement, the distal end of the catheter 800 is retained in close proximity to the elevation of the patient's heart, and, for the most part, remains in such proximity independent of the patient's position.

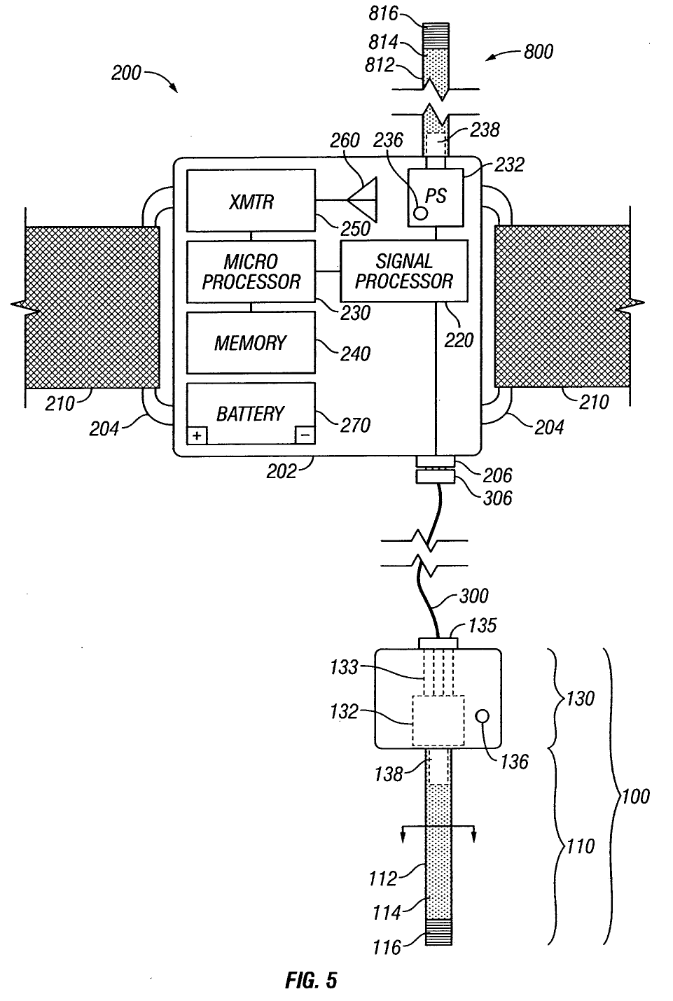

The proximal end of the catheter 800 may be either: (1) connected to a second (separate) pressure sensor 232 in the transmitter unit 200, as shown and described in more detail with reference to FIG. 5; or (2) may be connected to the back side of the pressure sensor 132 in the sensor portion 130. In the first embodiment, pressure measured by sensor 232, corresponding to pressure in close proximity to the heart as referred by the catheter 800, may be subtracted from vascular pressure measured by sensor 132 to correct for elevational differences. In the second embodiment, the vascular pressure measured by the front side of sensor 132 is automatically corrected for elevational differences because it is made relative to pressure measured by the back side of the sensor 132 corresponding to pressure in close proximity to the heart as referred by the catheter 800.

With reference to FIG. 5, details of the catheter 800 and the alternative transmitter unit 200 are schematically illustrated. In this alternative embodiment, transmitter unit 200 includes a pressure transducer 232 disposed in the housing 202, with a nipple tube 238 extending from the pressure transducer 232 and through the housing 202 for connection to the proximal end of the catheter 800. The pressure transducer 232 may be of the piezoresistive, resonant structure, or capacitive type, for example such as a piezoresistive Wheatstone bridge type silicon strain gauge available from Sensonor of Horten, Norway. The pressure transducer 232 is connected to signal processor 220 for performing signal processing functions such as filtering, A/D conversion, amplification, etc. A passage 236 such as a hole (shown) or flexible diaphragm may be used to provide pressure communication through the housing 202 such that the pressure transducer 232 may make pressure measurements relative to barometric pressure.

Catheter 800 refers pressure from a point in close proximity to the elevation of the patient's heart to the pressure transducer 232 located inside the housing 202. The catheter 800 may comprise a tubular shaft 812 filled with a pressure transmitting fluid 814 and including a distal barrier 816 such as a viscous plug (shown) or flexible membrane. The tubular shaft 812 of the catheter 800 may comprise, for example, a flexible polymeric tube and may incorporate an embedded coil or braid to prevent inadvertent kinking and/or collapse. The size (length and diameter) of the catheter 800 will vary depending on the distance from the transmitter unit 200 to the point proximate the elevation of the patient's heart. For example, the catheter 800 may have a diameter of approximately 1 mm-3 mm and a length of approximately 30 cm-50 cm to extend from a point near the radial artery at the wrist to a point on the upper arm near the level of the heart.

The barrier 816 may comprise a gel plug and/or flexible membrane disposed in or over the distal opening of the tubular shaft 812 to retain the fluid 814 in the lumen without impeding pressure transmission therethrough. To replicate the pressure head of blood created by elevational differences, the fill fluid may comprise a liquid with the same specific gravity as blood. If a liquid with a specific gravity other than that of blood is used, a correlation factor may be applied to the pressure measurement to replicate the pressure head of blood. In one embodiment, the fluid 114 may comprise a fluorinated silicone oil (which may require a correlation factor) and the barrier 116 may comprise a dimethyl silicone gel. The benefit of using a fluorinated silicone oil or other fluid comprised of a large molecule is that the fluid will not migrate from the lumen of the tube by either leaching or migration of vapors through the tubing wall. If the fluid was water or saline, for example, the volume of fluid 114 may shrink over time due to these factors, potentially resulting in an offset error.

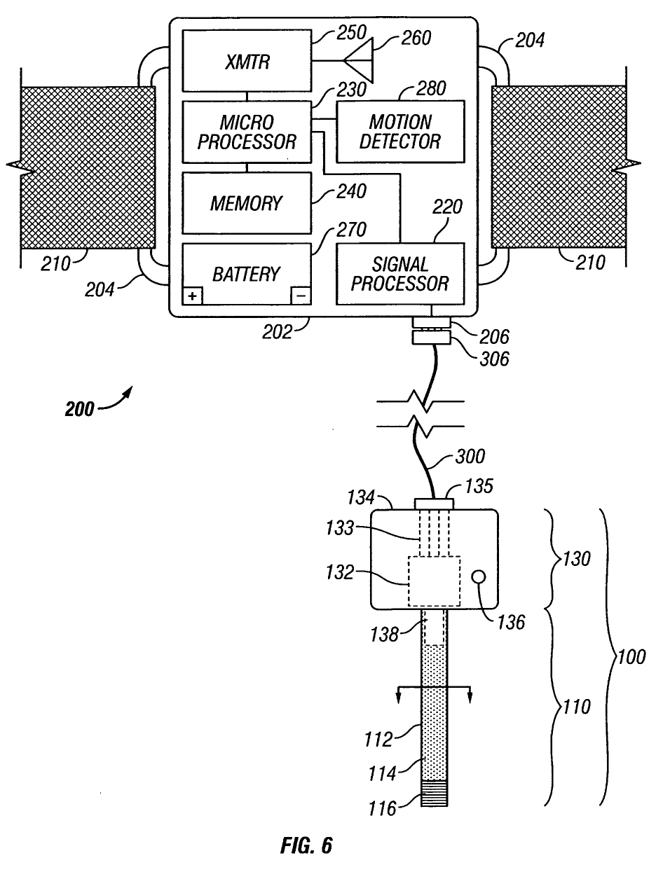

With reference to FIG. 6, an alternative approach to detecting elevational differences is shown schematically. In this approach, a motion detector 280 detects changes in patient position near the access site and flags pressure data that may have resulting artifacts. The motion detector 230 may be contained in the housing of the transmitter unit 200, and may comprise a level switch, an accelerometer, an activity sensor or the like known in the art. The motion detector 280 may be connected to the microprocessor 230 which flags pressure data upon an indication of movement. These flags are stored and/or displayed with the corresponding pressure data, thus enabling the attending nurse or physician to determine if pressure changes are due to an clinical change in blood pressure or are due to patient movement. This approach is particularly suitable for patients undergoing pressure monitoring in the supine position where the pressure measurement site is level with the heart, and changes in position will likely correspond to changes in relative elevation.

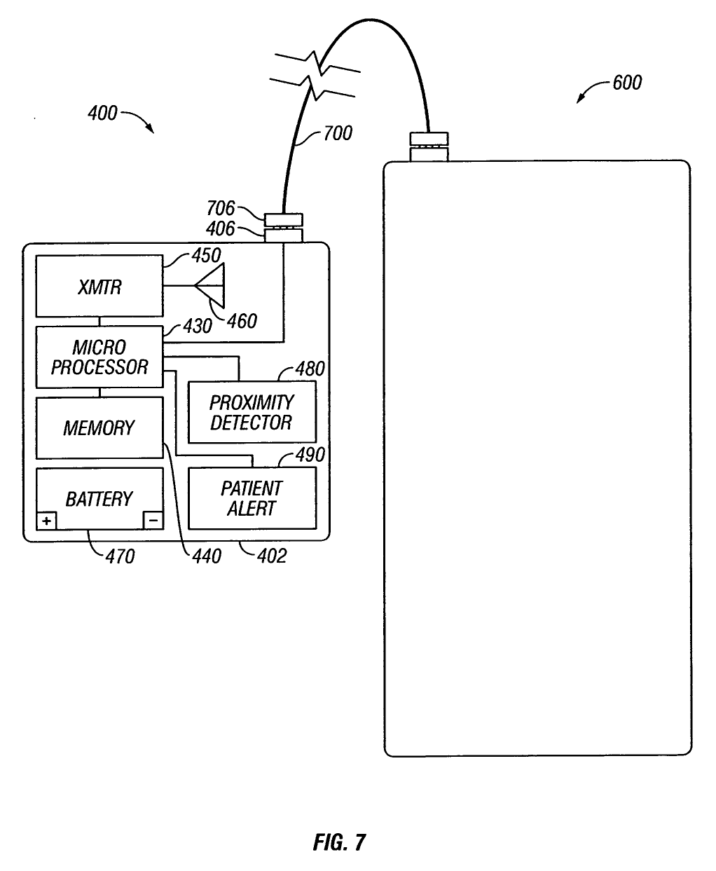

With reference to FIG. 7, another alternative approach to detecting elevational differences is shown schematically. In this approach, the receiver unit 400 is positioned proximate the level of the heart such as on the patient's chest near the heart. A patient alert 490 notifies the patient when a pressure monitoring session should be initiated, and the patient then moves the transmitter unit 200 and/or the sensor and catheter assembly 100 near the receiver unit 400. A proximity detector 480 may be used to detect the correct position of the transmitter unit 200 and/or the sensor and catheter assembly 100 near the receiver unit 400. With the receiver unit 400 near the heart, and the sensor and catheter assembly 100 near the receiver unit 400, elevational differences are substantially reduced if not eliminated. Vascular pressure may then be monitored without errors due to elevational differences. When the pressure monitoring period is over, the patient alert 490 notifies the patient that the pressure monitoring session has ended and that the transmitter unit 200 and/or the sensor and catheter assembly 100 may be returned to their original position.

The proximity detector 480 may be disposed in the housing 402 of the receiver unit 400, and connected to the microprocessor 430. The proximity detector 480 may comprise a variety of proximity detectors known in the art such as a reed switch activated by a corresponding magnet in the transmitter unit 200 or in the pressure sensor portion 130, for example. The patient alert 490 may also be disposed in the housing 402 of the receiver unit 400, and connected to the microprocessor 430. The patient alert 490 may comprise an audible, vibratory, and/or visible indicator to notify the patient when the pressure monitoring session begins and ends. By monitoring pressure only when the sensor and catheter assembly 100 is near the receiver unit 400 (which is near the heart), elevational errors may be minimized in the pressure readings.

From the foregoing, it will be apparent to those skilled in the art that the present invention provides, in exemplary no-limiting embodiments, systems and methods for monitoring intravascular pressure of a patient, with optional means for eliminating elevational error. Further, those skilled in the art will recognize that the present invention may be manifested in a variety of forms other than the specific embodiments described and contemplated herein. Accordingly, departures in form and detail may be made without departing from the scope and spirit of the present invention as described in the appended claims.

1. A system for monitoring intravascular pressure of a patient, comprising:
a pressure sensor and catheter assembly including a pressure sensor portion and a transdermal catheter portion, the transdermal catheter portion having a fluid filled lumen and a distal barrier;
a lead connected to the pressure sensor portion;
a transmitter unit releasably connected to the lead, the transmitter unit transmitting pressure data received from the pressure sensor via the lead;
a connector for releasable connection to the patient's body or clothing; and
a receiver unit for receiving pressure data from the transmitter unit.

2. A system as in claim 1, wherein the pressure sensor portion includes a pressure transducer, and wherein the pressure transducer has a minimum sensitivity of 5 uV/V/mmHg.

3. A system as in claim 1, wherein the transdermal catheter is configured to inserted through the patient's skin and into a blood vessel.

4. A system as in claim 3, wherein the blood vessel comprises a blood vessel in the patient's arm.

5. A system as in claim 4, wherein the blood vessel in the patient's arm comprises a radial artery.

6. A system as in claim 3, wherein the transdermal catheter has second lumen for drawing blood and/or infusing fluid.

7. A method for monitoring intravascular pressure of a patient, comprising:
providing a pressure sensor and catheter assembly including a pressure sensor portion and a transdermal catheter portion, the transdermal catheter portion having proximal end, a distal end, and a fluid filled lumen with a distal barrier;
inserting the transdermal catheter portion through the patient's skin and into a vascular lumen such that the distal end resides in the vascular lumen and the proximal end resides outside the skin;
providing a lead connected to the pressure sensor portion;
providing a transmitter unit having a means for connection to the patient's body or clothing;
releasably connecting the lead to the transmitter unit to receive pressure data from the pressure sensor portion;
connecting the transmitter unit to the patient's body or clothing;
providing a remote receiver unit; and
transmitting pressure data from the transmitter unit to the remote receiver unit.

8. A method as in claim 7, wherein the pressure sensor portion includes a pressure transducer, and wherein the pressure transducer has a minimum sensitivity of 5 uV/V/mmHg.

9. A method as in claim 7, wherein the blood vessel comprises a blood vessel in the patient's arm.

10. A method as in claim 9, wherein the blood vessel in the patient's arm comprises a radial artery.

11. A method as in claim 7, wherein the transdermal catheter has second lumen.

12. A method as in claim 11, further comprising the step of drawing blood through the second lumen.

13. A method as in claim 11, further comprising the step of infusing fluid through the second lumen.

14. A method for monitoring intravascular pressure of a human patient, comprising:
providing a pressure sensor and catheter assembly including a pressure sensor portion and a transdermal catheter portion, the transdermal catheter portion having a proximal end, a distal end, and a fluid filled pressure sensing lumen with a distal barrier, the pressure sensor portion connected to the proximal end of the catheter portion;
inserting the transdermal catheter portion through the patient' skin and into a blood vessel such that the distal end of the catheter portion resides in a lumen of the blood vessel and the proximal end of the catheter portion resides outside the skin; and
linking the pressure sensor portion to a monitoring instrument.

15. A method as in claim 14, further comprising:
providing a transmitter connected to and receiving a pressure signal from the pressure sensor portion; and
wirelessly transmitting pressure data representative of the pressure signal using the transmitter.

16. A method as in claim 14, wherein the step of linking the pressure sensor portion to the monitoring instrument comprises a hardwired link.

17. A method as in claim 16, wherein the pressure sensor portion includes a pressure transducer, and wherein the pressure transducer has a minimum sensitivity of 5 uV/V/mmHg.

18. A method as in claim 17, wherein the pressure transducer has a pressure range of −30 to 300 mmHg.

19. A method as in claim 18, wherein the pressure transducer has an excitation impedance greater than 200 Ohms.

20. A method as in claim 19, wherein the pressure transducer has an unbalance of less than ±75 mmHg.

21. A method as in claim 20, wherein the pressure transducer has an accuracy of 1 mmHg+1% of reading for −20 to 50 mmHg and ±3% of reading for 50 to 300 mmHg.

22. A method as in claim 17, wherein the blood vessel comprises a blood vessel in the patient's arm.

23. A method as in claim 22, wherein the blood vessel in the patient's arm comprises a radial artery.

24. A method as in claim 17, wherein the transdermal catheter has second lumen.

25. A method as in claim 24, further comprising the step of drawing blood through the second lumen.

26. A method as in claim 24, further comprising the step of infusing fluid through the second lumen.
